(19) United States
(12) Patent Application Publication
(10) Pub. No.: US 2013/0197071 A1
(43) Pub. Date: Aug. 1, 2013

(54) PRODUCTION OF LIPOXYGENASE INHIBITORS VIA FUNGAL BIOSYNTHETIC PATHWAY

(71) Applicant: University of Southern California, Los Angeles, CA (US)

(72) Inventors: Clay Wang, San Marino, CA (US); Amber D. Somonza, Bellflower, CA (US); Yi-Ming Chiang, Temple City, CA (US); Berl R. Oakley, Lawrence, KS (US)

(73) Assignee: UNIVERSITY OF SOUTHERN CALIFORNIA, Los Angeles, CA (US)

(21) Appl. No.: 13/752,276

(22) Filed: Jan. 28, 2013

Related U.S. Application Data
(60) Provisional application No. 61/591,764, filed on Jan. 27, 2012.

(51) Int. Cl. C07D 311/76

(52) U.S. Cl. CPC C07D 311/76
USPC 514/456; 549/409

(57) ABSTRACT

The invention provides methods for producing lipoxygenase inhibitors including the steps set forth in Schemes 2 and 3, and uses of the inhibitors produced by the methods set forth herein in to treat various disease states.

### CROSS-REFERENCE TO RELATED APPLICATION

This application claims priority to U.S. Ser. No. 61/591,764 filed Jan. 27, 2012, the contents of which are herein incorporated by reference in its entirety.

### GOVERNMENT RIGHTS

This invention was made with government support under Grant No. GM084077 awarded by the National Institutes of General Medical Sciences. The government has certain rights to the invention.

### FIELD OF INVENTION

The invention provides methods for producing lipoxygenase inhibitors and methods for treating diseases using the lipoxygenase inhibitors.

### BACKGROUND

Lipoxygenases are ubiquitous enzymes widely distributed within plants, fungi and mammals.¹ They are non-heme iron dioxygenases that catalyze the addition of molecular oxygen to polyunsaturated fatty acids with a cis, cis-1,4 pentadiene to generate a hydroperoxydiene formed through a radical, regio- and stereoselective mechanism.² Reaction products of lipoxygenase are involved in several common human disorders such as allergies, asthma, cancer, osteoporosis and other inflammatory diseases. Lipoxygenases are responsible for the oxidation lipids in foods subsequently reducing the foods' nutritional value.³

Several natural products from microbial sources inhibit 15-lipoxgyenase (15-LOX).⁴ More recently the fungal pigment, (+)-sclerotiorin (1) was found to inhibit lipoxygenase-1, also known as 15-LOX^{5,6} and has been found to inhibit multiple therapeutic targets.'

Sclerotiorin belongs to an important class of natural products called azaphilones. Azaphilones are structurally diverse polyketides that share a highly oxygenated bicyclic core and chiral quaternary center. These polyketides are known for their 4H-pyran motif, which reacts with amines to produce the corresponding vinylogous α-pyridones.⁸ Early synthetic studies by Whalley and coworkers reported the total synthesis of several azaphilones, which included compound 8 prepared in 14 steps.⁹ Recent synthetic efforts by Porco and coworkers have shown assembly of the azaphilone core through a copper-mediated enantioselective dearomatization route. The application of their asymmetric methodology was demonstrated on (−)—S-15183a (2), (−)-mitrorubin (3), and more recently with 1 (FIG. 1).¹⁰

Although many azaphilones have been isolated and identified, their biosynthetic pathways remained unknown until our recent identification of the asperfuranone (4) biosynthetic pathway in Aspergillus nidulans.¹¹ A mutant strain from the previous study provided aldehyde 5 as a stable intermediate, which has been isolated from other azaphilone-producing organisms. Herein, the inventors describe methods for producing the putative azaphilone intermediate (5) and use synthetic chemistry to structurally diversify 5 into natural and non-natural azaphilones.

### SUMMARY OF THE INVENTION

The invention provides methods for producing lipoxygenase inhibitors. The methods comprise the steps set forth in FIG. 3 (Scheme 2) and FIG. 4 (Scheme 3). In various embodiments, the aforementioned methods yield lipoxygenase inhibitors 7, 8, 9, 10, 11, 12, 13, 14, 15, 16 and 17 as set forth in FIGS. 3 and 4. The lipoxygenase inhibitors produced by the methods described herein may be used to treat and/or inhibit inflammatory diseases.

### BRIEF DESCRIPTION OF THE FIGURES

FIG. 1 depicts, in an embodiment of the invention, the natural products of azaphilone.

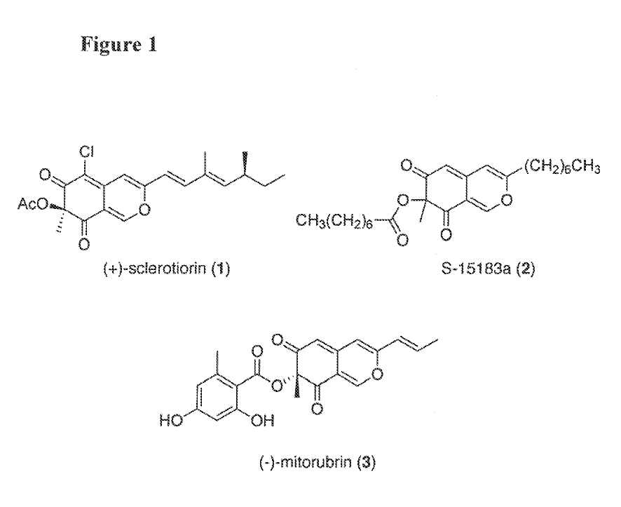

FIG. 2 depicts, in an embodiment of the invention, the reengineered biosynthetic pathway for the synthesis of (+)-sclerotiorin and 7-epi-sclerotiorin (8) and non-natural azaphilone polyketides (Scheme 1).

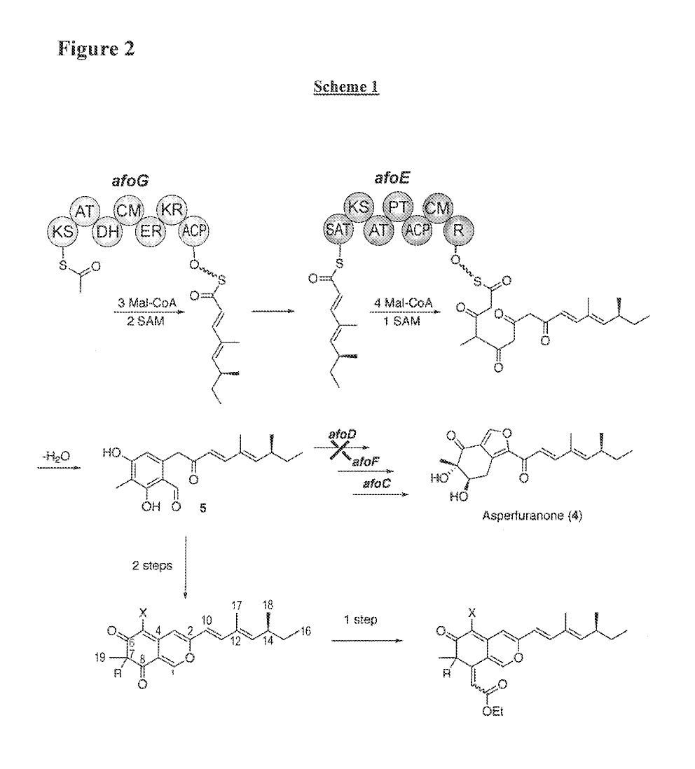

FIG. 3 depicts, in an embodiment of the invention, the concise synthesis of (+)sclerotiorin, 7-epi-sclerotiorin (8) and analogs (Scheme 2).

FIG. 4 depicts, in an embodiment of the invention, a short route to azaphilone analogs (Scheme 3).

FIG. 5 depicts, in an embodiment of the invention, (a) optimization of induction time and (b) optimization of culture time (post-induction).

FIG. 6 depicts, in an embodiment of the invention, a schematic of soybean lipoxygenase-1 assay.

FIG. 7 depicts, in an embodiment of the invention, (a) LOX-1 enzyme kinetics with varying concentrations of (+)-sclerotiorin measured at λ=234 nm, and (b) initial velocity (Vₒ) of LOX-1 vs. concentration of the inhibitor (+)-sclerotiori, where Vₒ of enzyme incubated in the absence of inhibitor designated a value of 1.

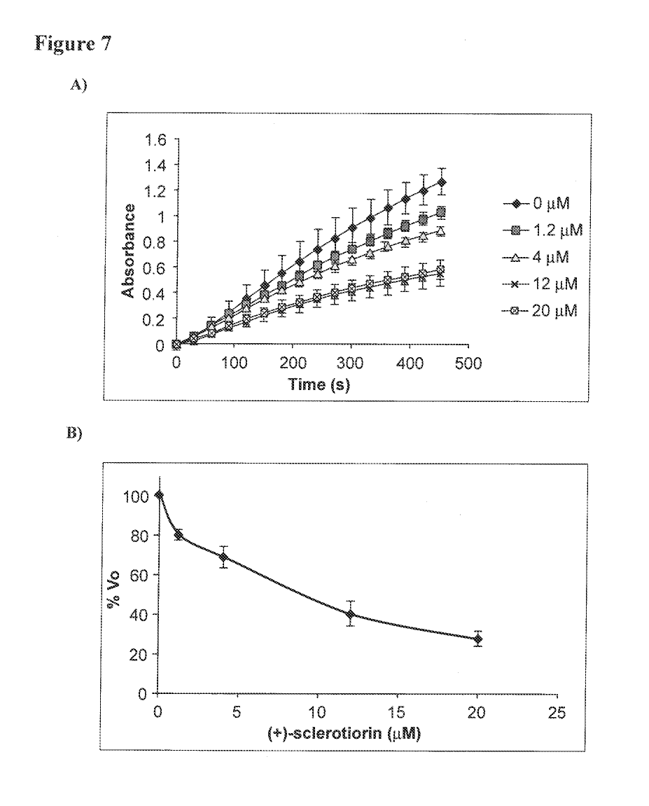

FIG. 8 depicts, in an embodiment of the invention, (a) LOX-1 enzyme kinetics with varying concentrations of compound 4 measured at λ=234 nm, and (b) initial velocity (Vₒ) of LOX-1 vs. concentration of the inhibitor compound 4, where Vₒ of enzyme incubated in the absence of inhibitor designated a value of 1.

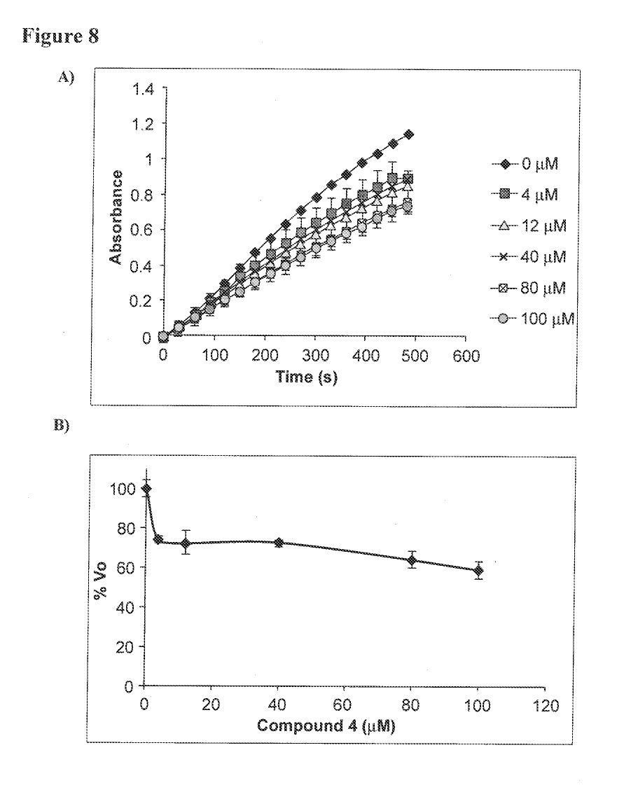

FIG. 9 depicts, in an embodiment of the invention, (a) LOX-1 enzyme kinetics with varying concentrations of compound 5 measured at λ=234 nm, and (b) initial velocity (Vₒ) of LOX-1 vs. concentration of the inhibitor compound 5, where Vₒ of enzyme incubated in the absence of inhibitor designated a value of 1.

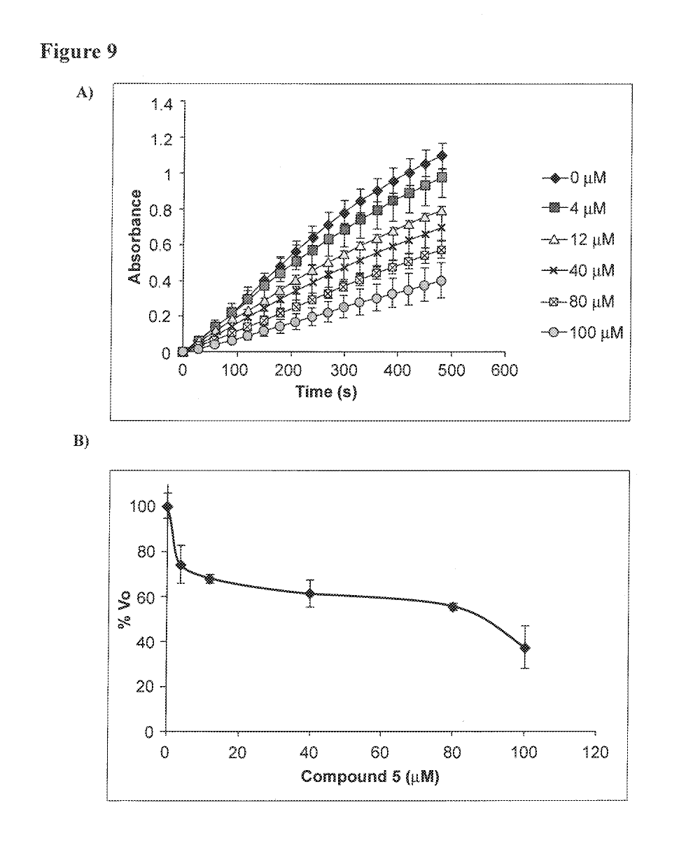

FIG. 10 depicts, in an embodiment of the invention, (a) LOX-1 enzyme kinetics with varying concentrations of compound 7 measured at λ=234 nm, and (b) initial velocity (Vₒ) of LOX-1 vs. concentration of the inhibitor compound 7, where Vₒ of enzyme incubated in the absence of inhibitor designated a value of 1.

FIG. 11 depicts, in an embodiment of the invention, (a) LOX-1 enzyme kinetics with varying concentrations of compound 8 measured at λ=234 nm, and (b) initial velocity (Vₒ) of LOX-1 vs. concentration of the inhibitor compound 8, where Vₒ of enzyme incubated in the absence of inhibitor designated a value of 1.

FIG. 12 depicts, in an embodiment of the invention, (a) LOX-1 enzyme kinetics with varying concentrations of compound 9 measured at λ=234 nm, and (b) initial velocity (Vₒ) of LOX-1 vs. concentration of the inhibitor compound 9, where Vₒ of enzyme incubated in the absence of inhibitor designated a value of 1.

FIG. 13 depicts, in an embodiment of the invention, (a) LOX-1 enzyme kinetics with varying concentrations of compound 10 measured at λ=234 nm, and (b) initial velocity (Vₒ) of LOX-1 vs. concentration of the inhibitor compound 10, where Vₒ of enzyme incubated in the absence of inhibitor designated a value of 1.

FIG. 14 depicts, in an embodiment of the invention, (a) LOX-1 enzyme kinetics with varying concentrations of compound II measured at λ=234 nm, and (b) initial velocity (Vₒ) of LOX-1 vs. concentration of the inhibitor compound II, where Vₒ of enzyme incubated in the absence of inhibitor designated a value of 1.

FIG. 15 depicts, in an embodiment of the invention, (a) LOX-1 enzyme kinetics with varying concentrations of compound 12 measured at λ=234 nm, and (b) initial velocity (Vₒ) of LOX-1 vs. concentration of the inhibitor compound 12, where Vₒ of enzyme incubated in the absence of inhibitor designated a value of 1.

FIG. 16 depicts, in an embodiment of the invention, (a) LOX-1 enzyme kinetics with varying concentrations of compound 13 measured at λ=234 nm, and (b) initial velocity (Vₒ) of LOX-1 vs. concentration of the inhibitor compound 13, where Vₒ of enzyme incubated in the absence of inhibitor designated a value of 1.

FIG. 17 depicts, in an embodiment of the invention, (a) LOX-1 enzyme kinetics with varying concentrations of compound 14 measured at λ=234 nm, and (b) initial velocity (Vₒ) of LOX-1 vs. concentration of the inhibitor compound 14, where Vₒ of enzyme incubated in the absence of inhibitor designated a value of 1.

FIG. 18 depicts, in an embodiment of the invention, (a) LOX-1 enzyme kinetics with varying concentrations of compound 15 measured at λ=234 nm, and (b) initial velocity (Vₒ) of LOX-1 vs. concentration of the inhibitor compound 15, where Vₒ of enzyme incubated in the absence of inhibitor designated a value of 1.

FIG. 19 depicts, in an embodiment of the invention, (a) LOX-1 enzyme kinetics with varying concentrations of compound 16 measured at λ=234 nm, and (b) initial velocity (Vₒ) of LOX-1 vs. concentration of the inhibitor compound 16, where Vₒ of enzyme incubated in the absence of inhibitor designated a value of 1.

FIG. 20 depicts, in an embodiment of the invention, (a) LOX-1 enzyme kinetics with varying concentrations of compound 17 measured at λ=234 nm, and (b) initial velocity (Vₒ) of LOX-1 vs. concentration of the inhibitor compound 17, where Vₒ of enzyme incubated in the absence of inhibitor designated a value of 1.

### DETAILED DESCRIPTION OF THE INVENTION

All references cited herein are incorporated by reference in their entirety as though fully set forth. The following description includes information that may be useful in understanding the present invention. It is not an admission that any of the information provided herein is prior art or relevant to the presently claimed invention, or that any publication specifically or implicitly referenced is prior art. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

Described herein are rapid semisynthetic methods to produce structurally related lipoxygenase inhibitors using an advanced aromatic polyketide intermediate 5 produced from a mutant strain of Aspergillus nidulans that allows synthesis of complex natural products in two to three synthetic organic steps.

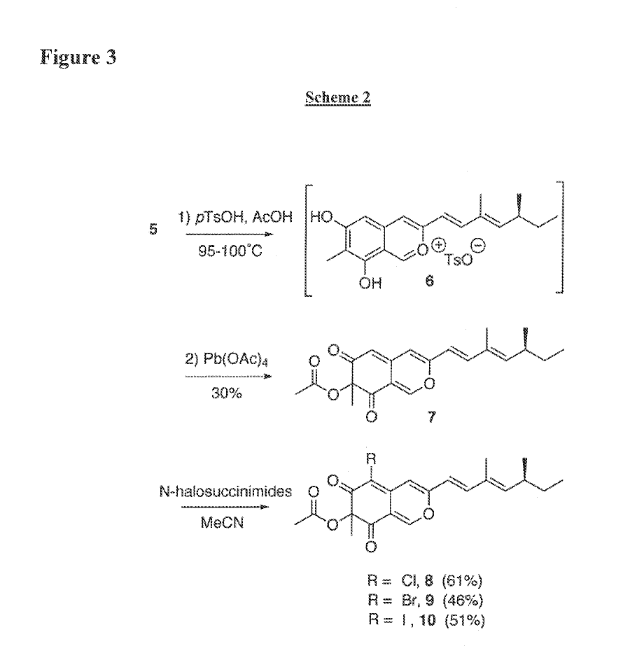

The invention provides a method for producing lipoxygenase (LOX) inhibitors. In some embodiments, the lipoxygenase inhibitor is an azaphilone or analogs thereof. In an embodiment, the azaphilone is sclerotiorin or analogs thereof. In one embodiment, lipooxygenase inhibitors are produced according to Scheme 2 set forth in FIG. 3. The method includes obtaining a compound 5 having the structure

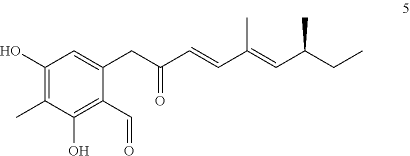 [SMILES: CC[C@H](C)/C=C(C)/C=C/C(=O)Cc1cc(O)c(C)c(O)c1C=O]

contacting the compound 5 with p-toluenesulfnic acid to obtain a compound 6 having the structure

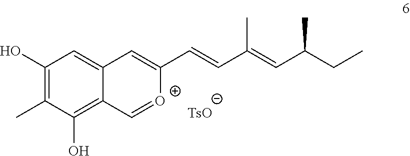 [SMILES: CC[C@H](C)/C=C(C)/C=C/c1cc2cc(O)c(C)c(O)c2c[o+]1]

contacting the compound 6 with an oxidizing agent to obtain compound 7 having the structure

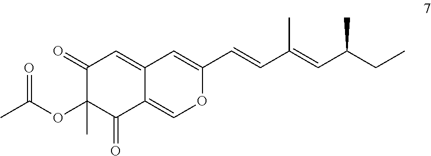 [SMILES: CC[C@H](C)/C=C(C)/C=C/C1=CC2=CC(=O)C(C)(OC(C)=O)C(=O)C2=CO1]

and, contacting the compound 7 with an N-halosuccinimide to obtain a compound having the structure

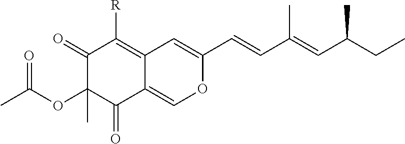

wherein R is Cl, Br or I and wherein the compound produced is LOX inhibitor. In some embodiments, an N-halosuccinimide includes but is not limited to any one or more of N-Chlorosuccinimide (NCS), N-Bromosuccinimide (NBS), N-Iodosuccinimide (NIS) or a combination thereof. In an embodiment, intermediate compound 5 is produced from a mutant strain of Aspergillus nidulans. In some embodiments, the oxidizing agent is lead tetracetate. In various embodiments, the lipoxygenase inhibitors produced by the methods described herein inhibit any one or more of 5-LOX, 8-LOX, 12-LOX, 15-LOX (including 15-LOX-1 and/or 15-LOX-2), or a combination thereof. In some embodiments, the aforementioned method yields compounds 7, 8, 9 and 10 as set forth in FIG. 3 (scheme 2).

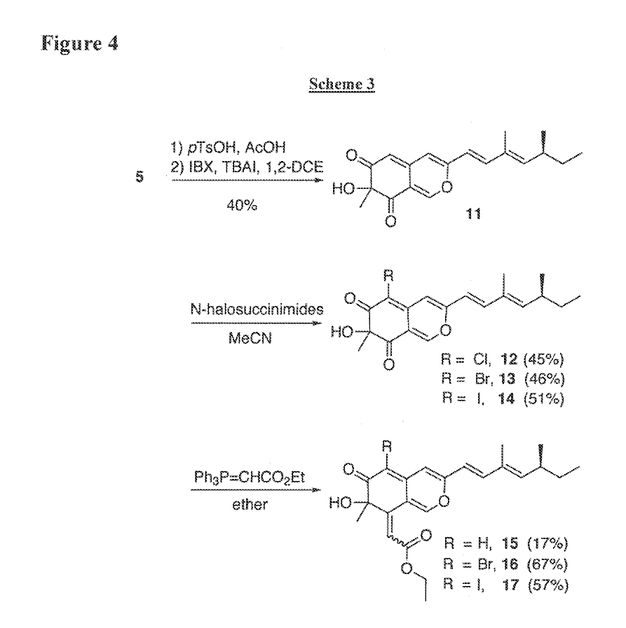

The invention provides an additional method for producing lipoxygenase (LOX) inhibitors. In some embodiments, the lipoxygenase inhibitor is an azaphilone or analogs thereof. In an embodiment, the azaphilone is sclerotiorin or analogs thereof. In one embodiment, lipooxygenase inhibitors are produced according to Scheme 3 set forth in FIG. 4. In some embodiments, an N-halosuccinimide includes but is not limited to any one or more of N—Chlorosuccinimide (NCS), N-Bromosuccinimide (NBS), N-Iodosuccinimide (NIS) or a combination thereof. In an embodiment, intermediate compound 5 is produced from a mutant strain of Aspergillus nidulans. In some embodiments, the oxidizing agent is lead tetracetate. In various embodiments, the lipoxygenase inhibitor inhibits any one or more of 5-LOX, 8-LOX, 12-LOX, 15-LOX (including 15-LOX-1 and/or 15-LOX-2), or a combination thereof. In some embodiments, the aforementioned method yields compounds 11, 12, 13, 14, 15, 16 and 17 as set forth in FIG. 4 (scheme 3).

The inventions provides compositions comprising lipoxygenase inhibitors produced by the methods described herein, as set forth in FIG. 3 (Scheme 2) and FIG. 4 (Scheme 3). In various embodiments, the composition includes any one or more of compounds 7, 8, 9, 10 as set forth in FIG. 3 (scheme 2) and/or compounds 11, 12, 13, 14, 15, 16 and 17 as set forth in FIG. 4 (scheme 3).

The invention further provides methods for treating inflammatory diseases in a subject in need thereof. The method includes providing a composition comprising a lipoxygenase inhibitor produced by the methods of the invention and administering an effective amount of the composition to the subject so as to treat inflammatory diseases in the subject. In various embodiments, the methods of the invention that produce lipoxygenase inhibitors are set forth in FIG. 3 (Scheme 2) and FIG. 4 (Scheme 3). In some embodiments, the composition includes any one or more of compounds 7, 8, 9, 10 as set forth in FIG. 3 (scheme 2) and/or compounds 11, 12, 13, 14, 15, 16 and 17 as set forth in FIG. 4 (scheme 3). The compositions of the invention may be administered alone or in conjunction with existing therapies. If other therapies are used in conjunction, the compositions of the invention may be administered concurrently or sequentially with the other the existing therapies.

The invention further provides methods for inhibiting inflammatory diseases in a subject in need thereof. The method includes providing a composition comprising a lipoxygenase inhibitor produced by the methods of the invention and administering an effective amount of the composition to the subject so as to inhibit inflammatory diseases in the subject. In various embodiments, the methods of the invention that produce lipoxygenase inhibitors are set forth in FIG. 3 (Scheme 2) and FIG. 4 (Scheme 3). In some embodiments, the composition includes any one or more of compounds 7, 8, 9, 10 as set forth in FIG. 3 (scheme 2) and/or compounds 11, 12, 13, 14, 15, 16 and 17 as set forth in FIG. 4 (scheme 3). The compositions of the invention may be administered alone or in conjunction with existing therapies. If other therapies are used in conjunction, the compositions of the invention may be administered concurrently or sequentially with the other the existing therapies.

The invention further provides methods for reducing the symptoms of inflammatory diseases in a subject in need thereof. The method includes providing a composition comprising a lipoxygenase inhibitor produced by the methods of the invention and administering an effective amount of the composition to the subject so as to reduce the symptoms of inflammatory diseases in the subject. In various embodiments, the methods of the invention that produce lipoxygenase inhibitors are set forth in FIG. 3 (Scheme 2) and FIG. 4 (Scheme 3). In some embodiments, the composition includes any one or more of compounds 7, 8, 9, 10 as set forth in FIG. 3 (scheme 2) and/or compounds 11, 12, 13, 14, 15, 16 and 17 as set forth in FIG. 4 (scheme 3). The compositions of the invention may be administered alone or in conjunction with existing therapies. If other therapies are used in conjunction, the compositions of the invention may be administered concurrently or sequentially with the other the existing therapies.

The invention further provides methods for promoting the prophylaxis of inflammatory diseases in a subject in need thereof. The method includes providing a composition comprising a lipoxygenase inhibitor produced by the methods of the invention and administering an effective amount of the composition to the subject so as to promote prophylaxis of inflammatory diseases in the subject. In various embodiments, the methods of the invention that produce lipoxygenase inhibitors are set forth in FIG. 3 (Scheme 2) and FIG. 4 (Scheme 3). In some embodiments, the composition includes any one or more of compounds 7, 8, 9, 10 as set forth in FIG. 3 (scheme 2) and/or compounds 11, 12, 13, 14, 15, 16 and 17 as set forth in FIG. 4 (scheme 3). The compositions of the invention may be administered alone or in conjunction with existing therapies. If other therapies are used in conjunction, the compositions of the invention may be administered concurrently or sequentially with the other the existing therapies.

In various embodiments, inflammatory diseases that may be treated with the aforementioned compositions include but are not limited to any one or more of allergies, asthma, atherosclerosis, cancer, osteoporosis, Alzheimer's disease and other forms of senile dementia.

### Pharmaceutical Compositions of the Invention

In various embodiments, the present invention provides pharmaceutical compositions including a pharmaceutically acceptable excipient and a therapeutically effective amount of lipoxygenase inhibitor produced by the methods set forth herein (for example Schemes 2 and 3). In various embodiments, the composition includes any one or more of compounds 7, 8, 9, 10 as set forth in FIG. 3 (scheme 2) and/or compounds 11, 12, 13, 14, 15, 16 and 17 as set forth in FIG. 4 (scheme 3).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. In various embodiments, the pharmaceutical compositions according to the invention may be formulated for delivery via any route of administration. In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. In one embodiment of the present invention the inventive compositions are injected directly into the bone marrow of the mammal. The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000)

### EXAMPLES

Sclerotiorin, an azaphilone polyketide, is a bioactive natural product known to inhibit 15-lipoxygenase and many other biological targets. To readily access sclerotiorin and analogs, the inventors developed a 2-3 step semisynthetic route to produce a variety of azaphilones starting from an advanced, putative azaphilone intermediate (5) over-produced by an engineered strain of Aspergillus nidulans. The inhibitory activities of the semisynthetic azaphilones against 15-lipoxygenase were evaluated with several compounds displaying low micromolar potency.

### Example 1

¹H and ¹³C NMR spectra were recorded on a Varian Mercury Plus 400 spectrometer. Chemical shifts are reported in ppm relative to CDCl₃ (¹H, δ 7.26; ¹³C, δ 77.0) or acetone-d6 (¹H, δ 2.05; ¹³C, δ 30.0, 206.0). ¹H NMR data is reported as: chemical shift, multiplicity (s=singlet, d=doublet, t=triplet, q=quartet, m=multiplet), coupling constant, and integration. Infrared spectra were recorded on a JASCO FTIR-4100. Infrared frequencies are reported in reciprocal centimeters. High resolution electrospray ionization mass was obtained on an Agilent 6210 time of flight LC-MS. Reactions were monitored by analytical thin-layer chromatography on EMD silica gel-60^{F254} plates. Flash chromatography was performed on EMD silica gel 60, 70-230 mesh. All reagents were used without further purification unless otherwise noted. Reagents were purchased from Sigma-Aldrich, and Alfa Aesar. Diethyl ether (anhydrous) and 1,2-dichloroethane (anhydrous) were used directly from the bottle. Water sensitive reactions were performed under a nitrogen atmosphere with oven-dried glassware. Kinetic experiments for lipoxygenase inhibition were recorded on a Shimadzu UV-2401PC at 26° C. Materials for the lipoxygenase assay such as linoleic acid, 15-Lipoxygenase (soybean PO, and (+)-sclerotiorin were purchased from Cayman Chemicals.

### Optimization of Cyclopentanone Inducing Time

Five flasks inoculated with alcA(p)-afoA, afoDΔ A. nidulans containing 1×10⁶ spores per mL in a liquid lactose minimal medium (lactose 15 g, NaNO₃ 6 g, KCl 0.52 g, MgSO₄.7H2O 0.52 g, KH₂PO₄ 1.52 g, H₂O 1 L supplemented with 1 mL of trace elements and adjusted to a pH of 6.5) were incubated at 37° C. in a Barnstead/Lab-Line MaxQ 4000 rotary shaker at a speed of 180 rpm. After incubation for the times shown, the chemical inducer, cyclopentanone was added in a single dose to a final concentration of 30 mM. After induction, the strain was cultured for three days under the same conditions.

### Extraction and Isolation

The mycelium was collected by filtration and then immersed in 50 mL of acetone. The mycelium-acetone mixture was subjected to stirring for 0.5 h and filtered to isolate the organic solution. The solvent was removed in vacuo to leave a light yellow solid residue analyzed by LC-MS and ¹H NMR to be compound 5.

### Optimization of Culture Time

The A. nidulans strain, alcA(p)-afoA, afoDΔ, at a concentration of 1×10⁶ spores per mL was incubated 37° C. in 25 mL of liquid lactose minimal medium (lactose 15 g, NaNO₃ 6 g, KCl 0.52 g, MgSO₄.7H2O 0.52 g, KH₂PO₄ 1.52 g, H2O 1 L supplemented with 1 mL of trace elements and adjusted to a pH of 6.5) at a speed of 180 rpm. For induction, cyclopentanone at a final concentration of 30 mM was introduced to the medium at 30 h of incubation. The strain was cultured for several days, and the mycelia were collected from day 3 to day 7 and analyzed for production of 5.

TABLE 1
A. nidulans strain used in this study

| Strain | Genotype |
|---|---|
| LO2955 | pyrG89; pyroA4, nkuA::argB; riboB2, stcJ::riboB; |
| | afoA::AfpyrG-alcA(p)-afoA; afoD::AfpyroA |

afoA::AfpyrG-alcA(p)-afoA is a replacement of the endogenous promoter of afoA with the alcA promoter and the a. fumigatus pyrG gene (AfpyrG). AfpyroA is the A. fumigatus pyroA gene.

### Soybean Lipooxygenase-1 Assay

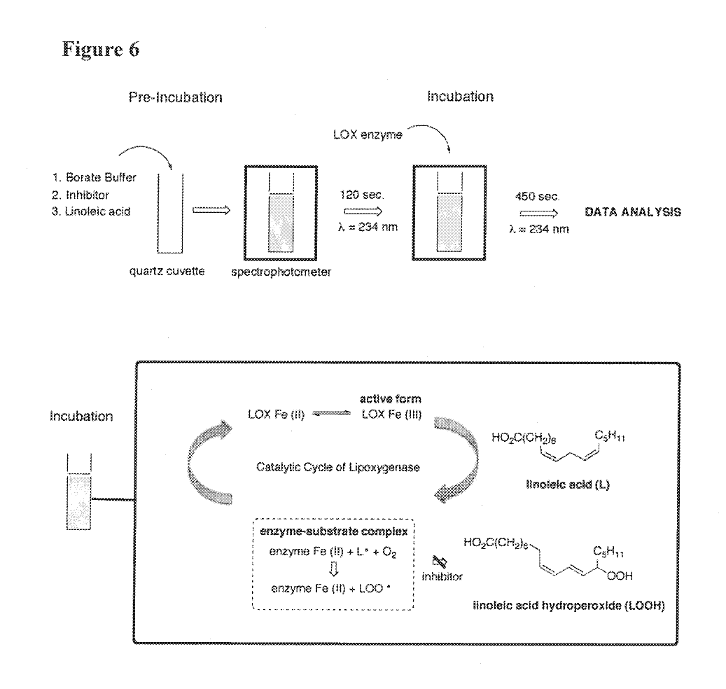

The enzyme assay was performed according to Axelrod et. al.¹⁸ Each solution was prepared and measured at room temperature. Enzyme kinetics was measured on a Shimadzu UV-2401PC at a wavelength of 234 nm in a quartz cuvette (FIG. 6). For the assay, the linoleic acid substrate was prepared by mixing 20 μL linoleic acid (Cayman Chemical #760716) with 20 μL KOH (Cayman Chemical #760713) and 40 μL double distilled water. Lipoxygenase-1 (Cayman Chemical #60712) was diluted from original stock concentration to a concentration of 100 U/mL in the cuvette. The semisynthetic azaphilones were prepared as stock concentrations in dimethylsulfoxide (DMSO) from 20 μM to 10 mM; 54, was added to the assay mixture to give the desired final concentration of the inhibitor with 1% of DMSO in the assay.

### Lipoxygenase-1 Inhibition Assay:

The spectrophotometric assay is based on monitoring the reaction, catalyzed by LOX-1 to convert linoleic acid (L) to the hydroperoxide (LOOH), at 234 nm. The substrate L does not absorb at the indicated wavelength, so that any absorption is due to the generation of LOOH. The reaction is initiated when the non-heme iron of LOX-1 in the ferric state is reduced by the substrate L. While in the enzyme-substrate complex, L is oxidized and converted to the peroxide free radical (LOO.), and subsequently released from the enzyme as LOOH Inhibitors can interfere in the process in several different ways to prevent the generation of LOOH by the chelation of iron, removal of oxygen or prevention of free radical formation.

Prior to measurements, a sample with buffer was used as a blank. In a quartz cuvette, each sample was prepared with 482.54, 0.2 M borate buffer (at a pH of 9.0) mixed with 54, inhibitor and 54, substrate by pipetting the solution. The cuvette was placed in the spectrophotometer and measured for 120 seconds and then rapidly 7.54, lipoxygenase-1 was added and mixed to initiate the reaction. Measurements were recorded every 30 seconds for 7.5 minutes.

IC₅₀ Calculation:

Inhibition of an enzyme at 50% can be experimentally measured by monitoring enzyme kinetics. A kinetic study generates a curve where the slope provides the initial velocity (Vₒ) of the enzyme. For each sample with different inhibitor concentrations the initial velocity was derived from a linear segment between 210-300 nm. The absorbance was used directly and not converted into concentration units. These data points (Vₒ, [Inhibitor]) were fitted to a curve using the software Curve Expert Professional 1.5.0 to provide an equation for the curve; the best mathematical model for the data was the MMF model. The concentration of inhibitor at which 50% inhibition occurred (as measured by change in Vₒ) was reported as the IC₅₀.

### Production of Compounds 7-17

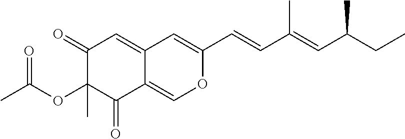 [SMILES: CC[C@H](C)/C=C(C)/C=C/C1=CC2=CC(=O)C(C)(OC(C)=O)C(=O)C2=CO1]

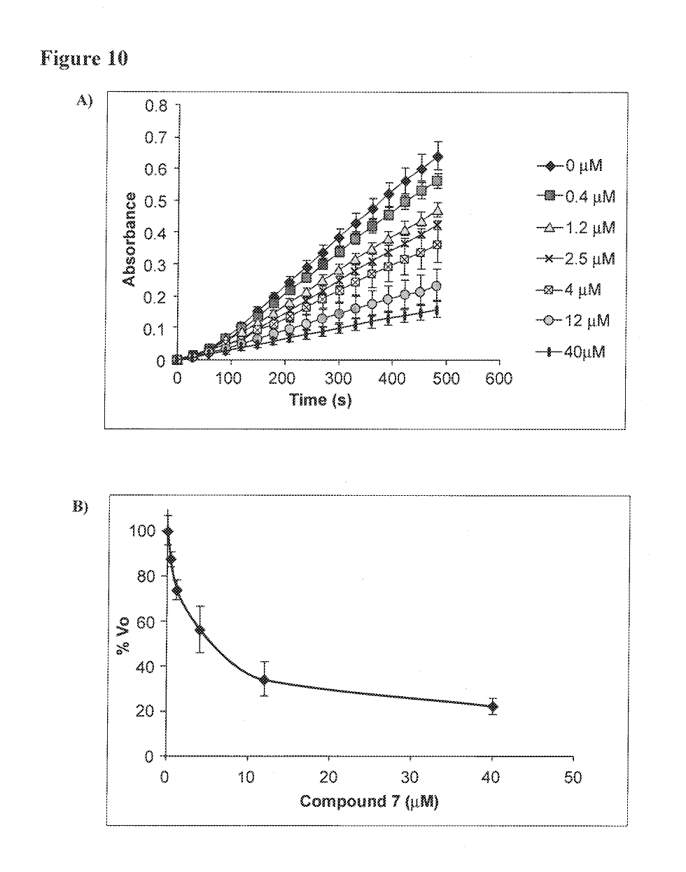

To 2,4-dihydroxy-6-(5,7-dimethyl-2-oxo-trans-3-trans-5-nonadienyl)-3-methylbenzaldehyde 5 (151 mg, 0.48 mmol) in 60 mL of acetic acid at room temperature was added in one portion p-TsOH.H₂O (940 mg, 4.94 mmol). The reaction was warmed in an oil bath to 100° C. and stirred for 2 hours. Then the reaction was cooled to room temperature and purged with nitrogen for 1.5 hours. It was further cooled to 18° C. and then treated with lead tetraacetate (276 mg, 0.621 mmol) in three portions over a 15-minute period. The solution stirred for 1 hour, and was quenched with 200 mL of ice water. The reaction mixture was extracted with dichloromethane 3×, and the combined organic layers were dried over magnesium sulfate, filtered and concentrated under reduced pressure. The crude product was purified by flash chromatography on silica gel (15% EtOAc/DCM) to afford 50.3 mg of compound 4. Isolated yield: 30%. The LOX-1 enzyme kinetics in the presence of compound 7 is shown in FIG. 10 and Table 2.

### Compound 8

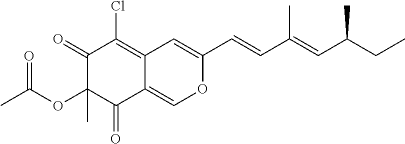 [SMILES: CC[C@H](C)/C=C(C)/C=C/C1=CC2=C(Cl)C(=O)C(C)(OC(C)=O)C(=O)C2=CO1]

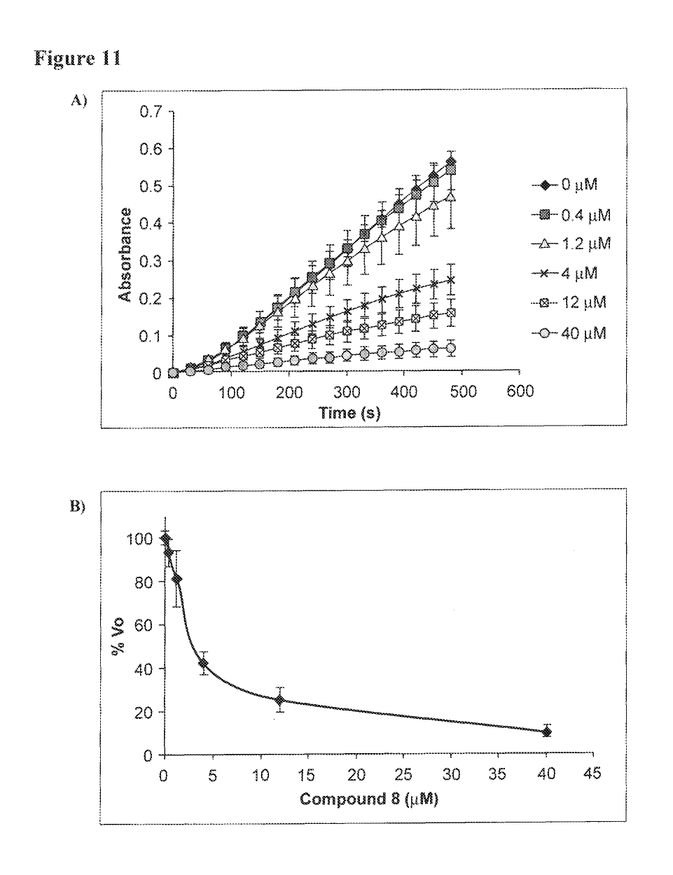

To a stirring solution of compound 7 (28.4 mg, 0.078 mmol) in acetic acid (0.93 mL) was added N-chlorosuccinimide (14 mg, 0.105 mmol) at room temperature. The solution stirred for 3 hours and then quenched with saturated Na₂S₂O₃ aqueous solution and diluted with ethyl acetate. The resulting mixture was extracted with ethyl acetate and the combined organic layers washed with NaHCO₃, water, and brine. It was dried over anhydrous sodium sulfate, filtered and concentrated under reduced pressure. The crude product was purified by flash chromatography on silica gel (25% EtOAc/n-hexanes) to afford 16 mg of compound 8. Isolated yield: 52%. The LOX-1 enzyme kinetics in the presence of compound 8 is shown in FIG. 11 and Table 2.

The diastereomers of 8 were separated by analytical chiral HPLC (Diacel CHIRALCEL® OD column 0.46×25 cm, 10% EtOH-hexanes, 1.0 mL/min, 360 nm, tR=7.5 min 7-epi-sclerotiorin, 9.5 min (+)-sclerotiorin compared with commercial available (+)-sclerotiorin.

### Compound 9

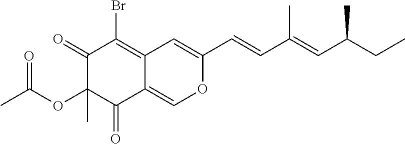 [SMILES: CC[C@H](C)/C=C(C)/C=C/C1=CC2=C(Br)C(=O)C(C)(OC(C)=O)C(=O)C2=CO1]

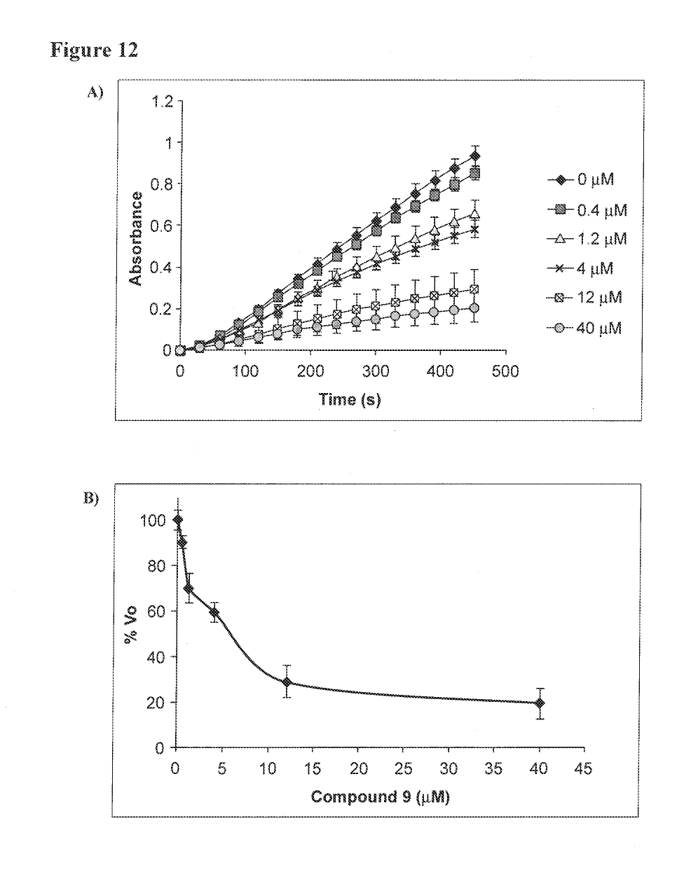

To a stirring solution of compound 7 (13.9 mg, 0.39 mmol) in acetonitrile (0.650 mL) was added N-bromosuccinimide (10 mg, 0.056 mmol) at room temperature. The solution stirred for 4 hours after which it was quenched with water and extracted with ethyl acetate. The organic layers were combined, washed with brine, dried over anhydrous sodium sulfate, filtered and concentrated under reduced pressure. The crude product was purified by flash chromatography on silica gel (20% EtOAc/n-hexanes) to afford 68.9 mg of compound 9. Isolated yield: 41%. The LOX-1 enzyme kinetics in the presence of compound 9 is shown in FIG. 12 and Table 2.

### Compound 10

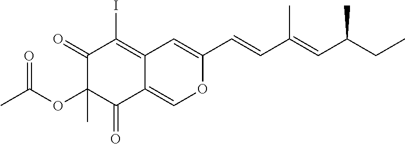 [SMILES: CC[C@H](C)/C=C(C)/C=C/C1=CC2=C(I)C(=O)C(C)(OC(C)=O)C(=O)C2=CO1]

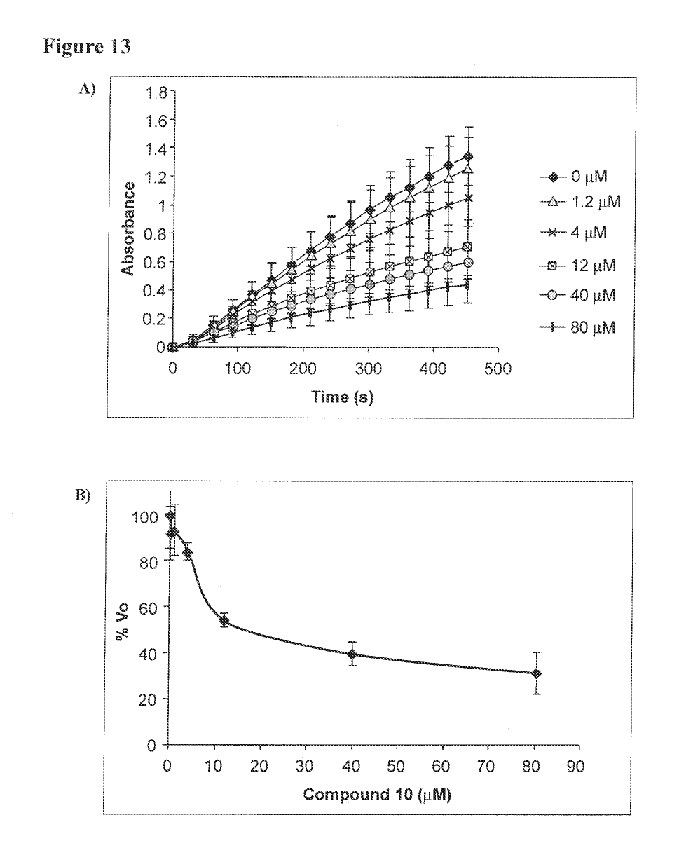

To a stirring solution of compound 7 (19.1 mg, 0.054 mmol) in acetonitrile (1.3 mL) was added N-iodosuccinimide (14 mg, 0.059 mmol) at room temperature. The solution stirred for 30 minutes and quenched with water. The mixture was extracted with ethyl acetate; combined organic layers were washed with brine, dried over anhydrous sodium sulfate, and concentrated under reduced pressure. Crude extract was purified by flash chromatography on silica gel (20% EtOAc/n-hexanes) to afford 7.2 mg of compound 10. Isolated yield: 28%. The LOX-1 enzyme kinetics in the presence of compound 10 is shown in FIG. 13 and Table 2.

### Compound 11

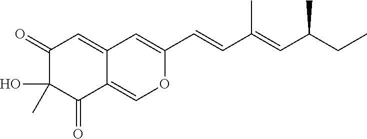 [SMILES: CC[C@H](C)/C=C(C)/C=C/C1=CC2=CC(=O)C(C)(O)C(=O)C2=CO1]

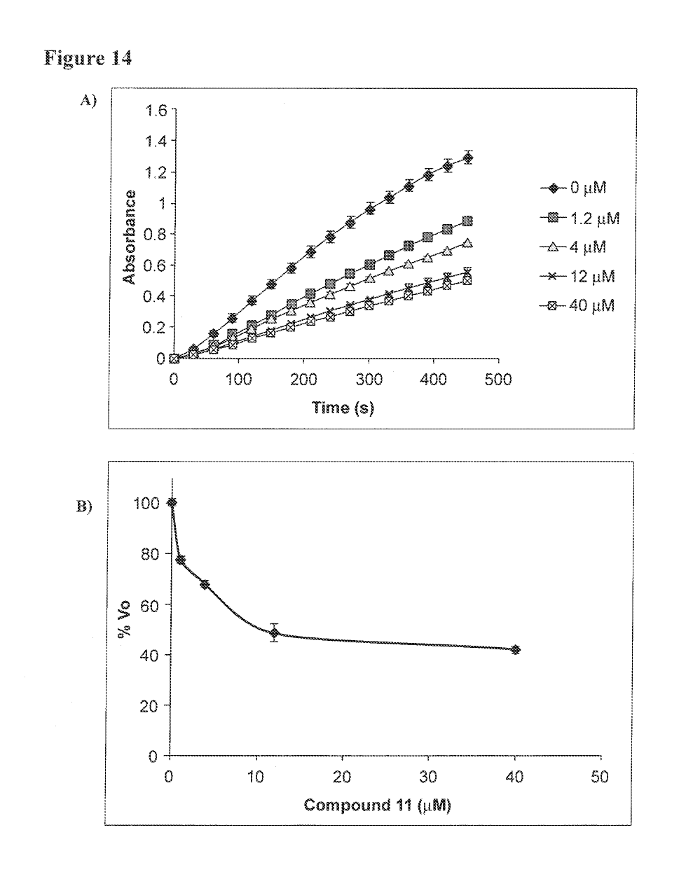

To 2,4-dihydroxy-6-(5,7-dimethyl-2-oxo-trans-3-trans-5-nonadienyl)-3-methylbenzaldehyde 5 (200 mg, 0.635 mmol) in 2.5 mL of acetic acid at room temperature was added p-TsOH (425 mg, 2.47 mmol). The suspension stirred for 1.5 hours under nitrogen leaving an orange precipitate, 2-benzopyrilium salt (6). The acetic acid was removed and the precipitate redissolved in 1,2-dichloroethane (5 mL) and then treated with freshly prepared 2-iodoxybenzoic acid (IBX)S3 (182 mg, 0.65 mmol), which was followed by the addition of tert-n-butyl ammonium iodide (23 mg, 0.062 mmol). The solution stirred for six hour and quenched with saturated NaS2O3 and ethyl acetate. The mixture was extracted with ethyl acetate 4×, and the combined organic layers were washed with saturated NaHCO3, water and brine. It was subsequently dried with anhydrous sodium sulfate, filtered and concentrated under reduced pressure. The crude product was purified by flash chromatography on silica gel (60% EtOAc/n-hexanes) to afford 79.4 mg of compound II. Isolated yield: 40%. The LOX-1 enzyme kinetics in the presence of compound II is shown in FIG. 14 and Table 2.

### Compound 12

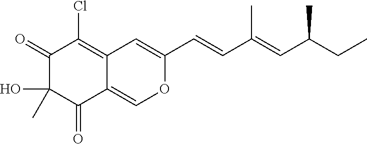 [SMILES: CC[C@H](C)/C=C(C)/C=C/C1=CC2=C(Cl)C(=O)C(C)(O)C(=O)C2=CO1]

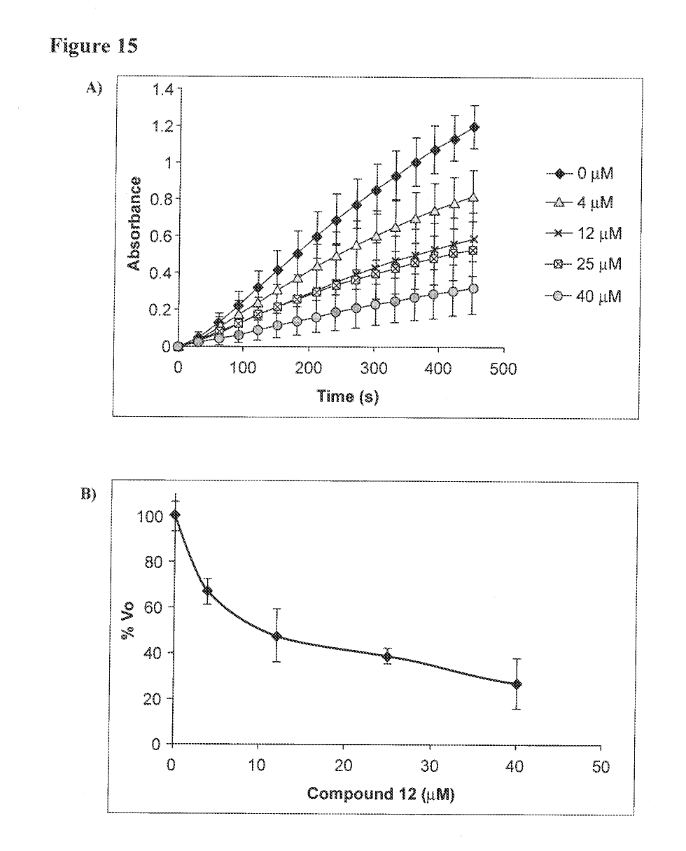

To a stirring solution of compound II (59.3 mg, 0.188 mmol) in 3.0 mL of acetonitrile at room temperature was added N-chlorosuccinimide (27 mg, 0.202 mmol). The solution stirred for 48 hours and quenched with water then extracted with ethyl acetate. The combined organic layers were washed with brine, dried over anhydrous sodium sulfate, filtered and concentrated under reduced pressure. Crude extract was purified by flash chromatography on silica gel (45% EtOAc/n-hexanes) to afford 29.3 mg of compound 12. Isolated yield: 45%. The LOX-1 enzyme kinetics in the presence of compound 12 is shown in FIG. 15 and Table 2.

### Compound 13

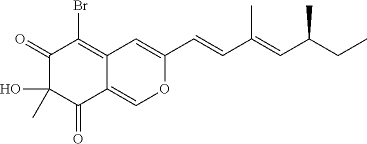 [SMILES: CC[C@H](C)/C=C(C)/C=C/C1=CC2=C(Br)C(=O)C(C)(O)C(=O)C2=CO1]

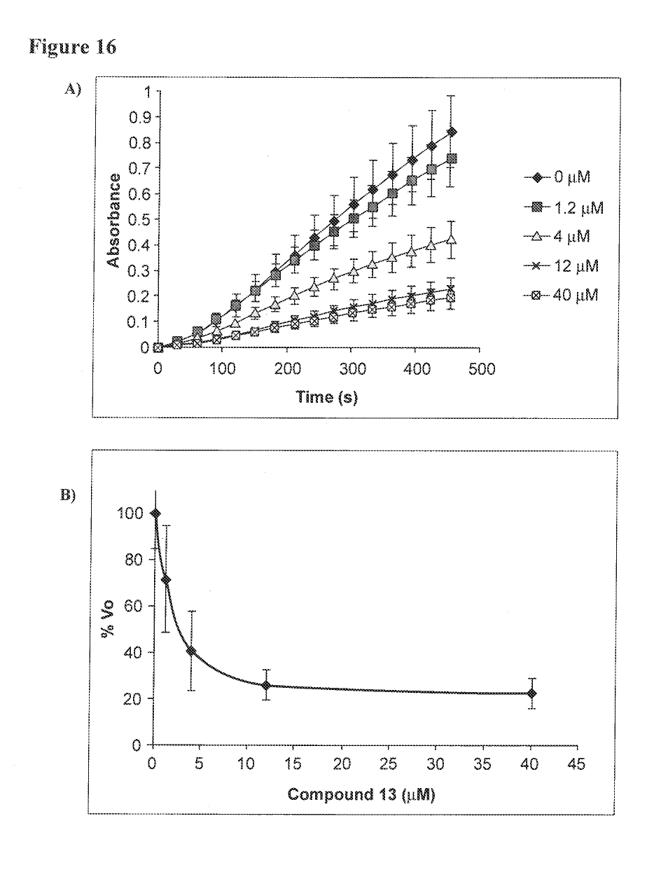

To a stirring solution of compound II (122.09 mg, 0.39 mmol) in acetonitrile (6.5 mL) was added N-bromosuccinimide (77.0 mg, 0.43 mmol) at room temperature. The solution stirred for 45 minutes and was then quenched with water. The mixture was extracted with ethyl acetate and the combined organic layers were dried over anhydrous sodium sulfate, filtered and concentrated under reduced pressure. The crude product was purified by flash chromatography on silica gel (20% EtOAc/n-hexanes) to afford 71 mg of compound 13. Isolated yield: 46%. The LOX-1 enzyme kinetics in the presence of compound 13 is shown in FIG. 16 and Table 2.

### Compound 14

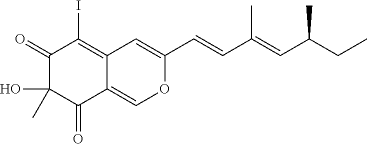 [SMILES: CC[C@H](C)/C=C(C)/C=C/C1=CC2=C(I)C(=O)C(C)(O)C(=O)C2=CO1]

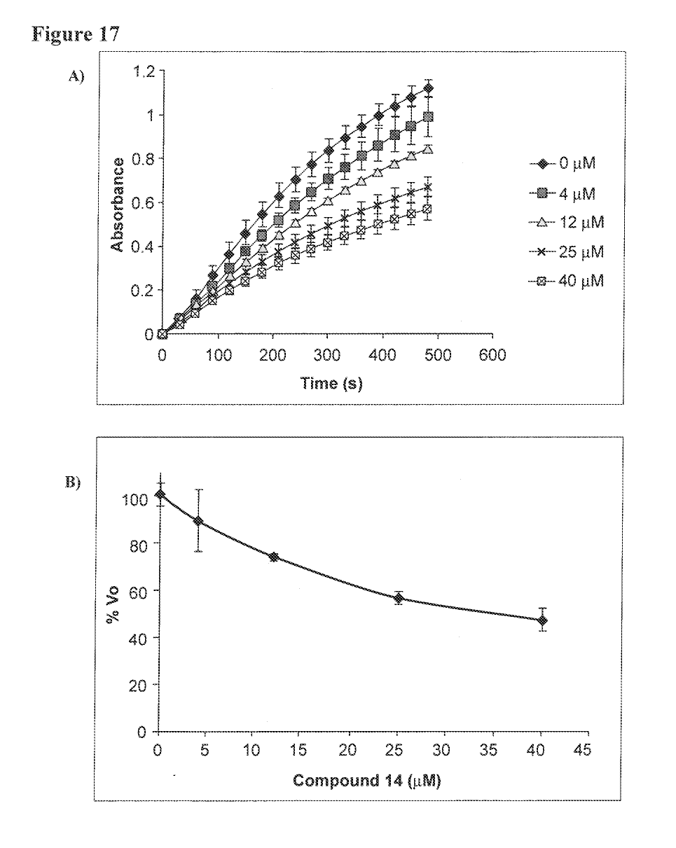

To a stirring solution of compound II (33.2 mg, 0.106 mmol) in acetonitrile (2.1 mL) was added N-iodosuccinimide (28 mg, 0.124 mmol) at room temperature. The solution stirred for 1 hour and quenched with water. The mixture was extracted with ethyl acetate; organic layers were combined and washed with brine, dried over anhydrous sodium sulfate and concentrated under reduced pressure. The crude product was purified by flash chromatography on silica gel (35% EtOAc/n-hexanes) to afford 23.7 mg of compound 14. Isolated yield: 51%. The LOX-1 enzyme kinetics in the presence of compound 14 is shown in FIG. 17 and Table 2.

### Compound 15

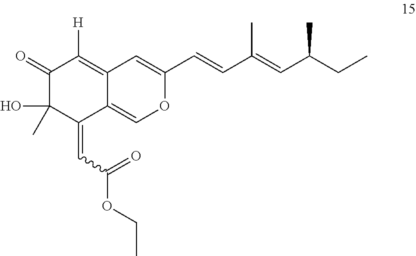 [SMILES: CCOC(=O)C=C1C2=COC(/C=C/C(C)=C/[C@@H](C)CC)=CC2=CC(=O)C1(C)O]

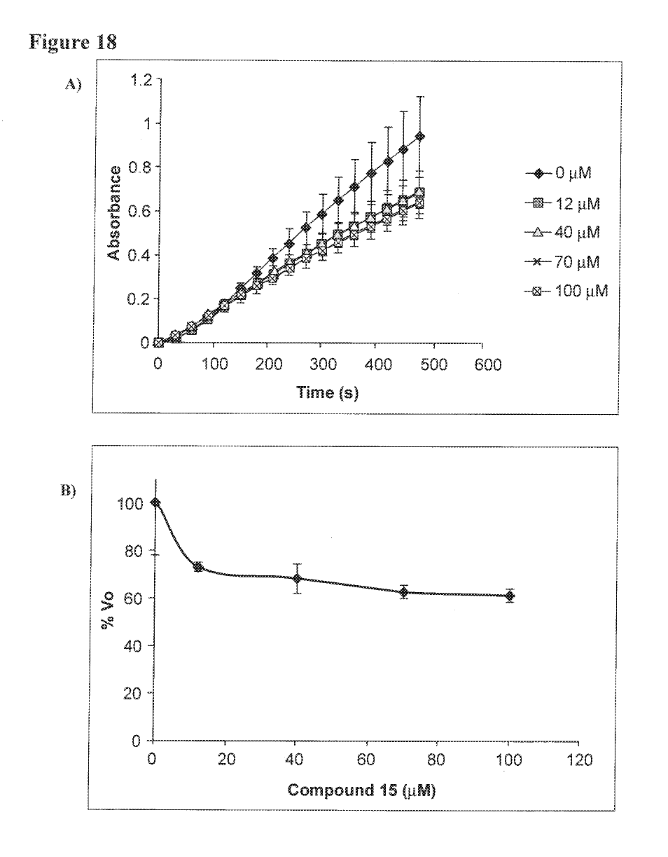

To a stirring solution of compound II (55 mg, 0.175 mmol) in anhydrous diethyl ether (6 mL) was added Wittig reagent, Ph3=CHCO2Et, (68 mg, 0.193 mmol). The solution stirred for 7 hours at room temperature. The solvent was removed under reduced pressure and crude extract purified by flash chromatography on silica gel (15% acetone/n-hexanes) to afford 11 mg of compound 15. Isolated yield: 17%. The LOX-1 enzyme kinetics in the presence of compound 15 is shown in FIG. 18 and Table 2.

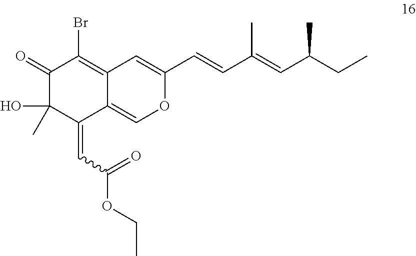 [SMILES: CCOC(=O)C=C1C2=COC(/C=C/C(C)=C/[C@@H](C)CC)=CC2=C(Br)C(=O)C1(C)O]

### Compound 16

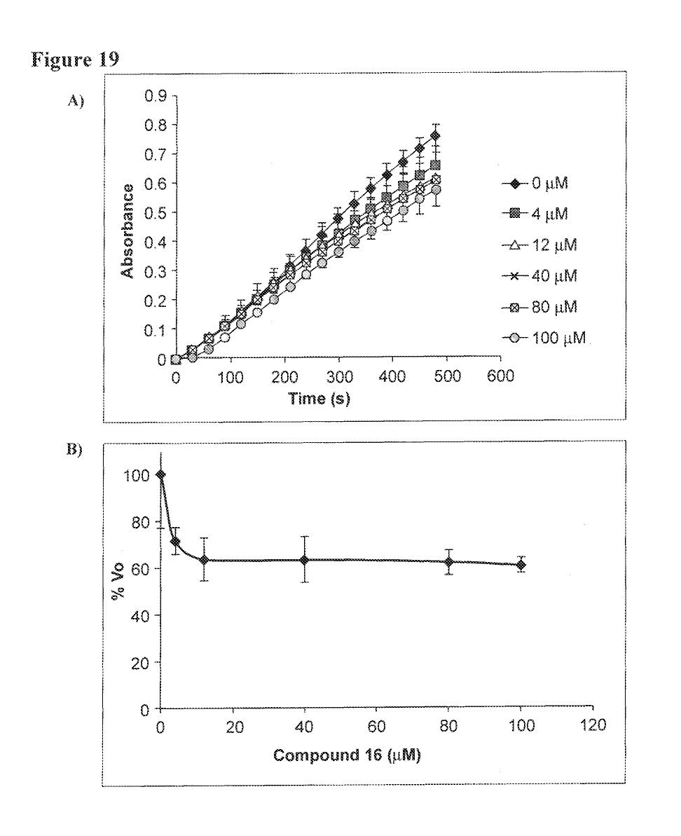

To a stirring solution of compound 13 (20.5 mg, 0.052 mmol) in anhydrous diethyl ether (2.6 mL) was added Ph3=CHCO2Et, (23 mg, 0.066 mmol). The solution stirred for 6 hours at room temperature. The solvent was removed under reduced pressure and crude extract purified by flash chromatography on silica gel (15% acetone/n-hexanes) to afford 16.3 mg of compound 16. Isolated yield: 68%. The LOX-1 enzyme kinetics in the presence of compound 16 is shown in FIG. 19 and Table 2.

### Compound 17

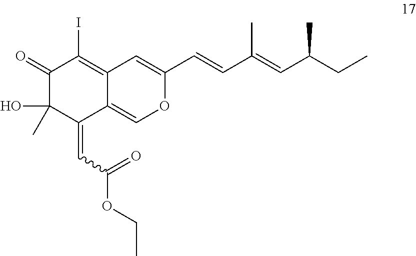 [SMILES: CCOC(=O)C=C1C2=COC(/C=C/C(C)=C/[C@@H](C)CC)=CC2=C(I)C(=O)C1(C)O]

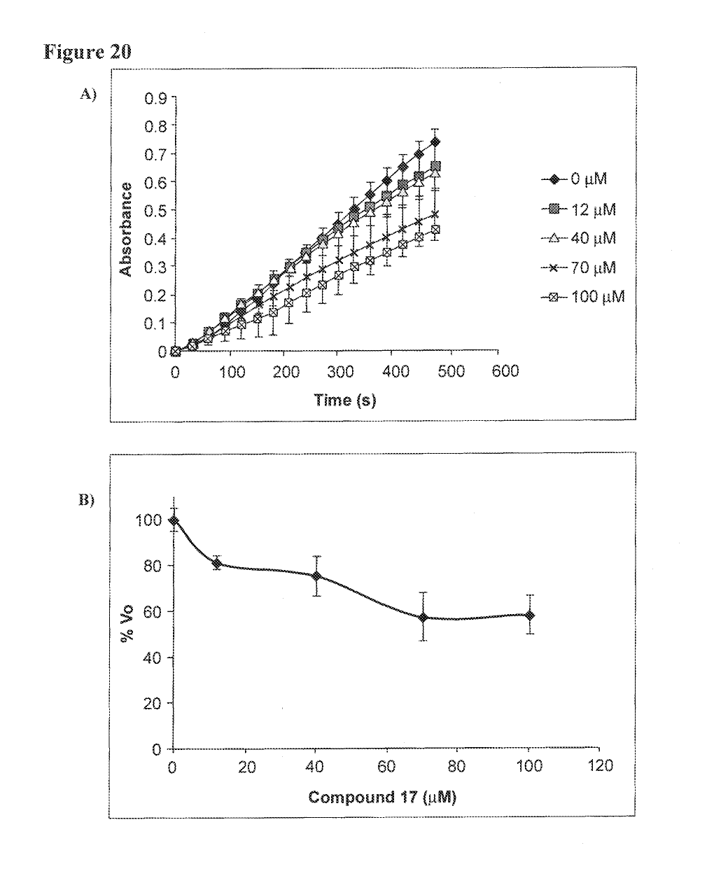

To a stirring solution of compound 14 (48 mg, 0.109 mmol) in diethyl ether (5.0 mL) was added Ph₃═CHCO₂Et, (46 mg, 0.132 mmol). The solution stirred for 6 hours at room temperature. The solvent was removed under reduced pressure and crude extract purified by flash chromatography on silica gel (15% acetone/nhexanes) to afford 31.8 mg of compound 17. Isolated yield: 57%. The LOX-1 enzyme kinetics in the presence of compound 17 is shown in FIG. 20 and Table 2.

### Example 2

The fungal strain used to over-produce compound 5 contains genetic alterations as described in Table 1. The native promoter of afoA, the gene that codes for the pathway-specific transcription activator of the asperfuranone pathway was replaced with the inducible alcohol dehydrogenase promoter, alcA. The afoD gene, that codes for the hydroxylase in the asperfuranone pathway, was deleted to enable the accumulation of intermediate, compound 5.¹¹ It should be noted that the afo cluster is silent under normal laboratory growing conditions. The wild type strain, thus, produced non-detectable compound 5 and asperfuranone (4). We initially cultured the strain in a liquid lactose minimal medium under inducing conditions at 37° C. for three days to produce nearly 200 mg/L of 5 without need for further purification since 5 is poorly dissolved in aqueous media.

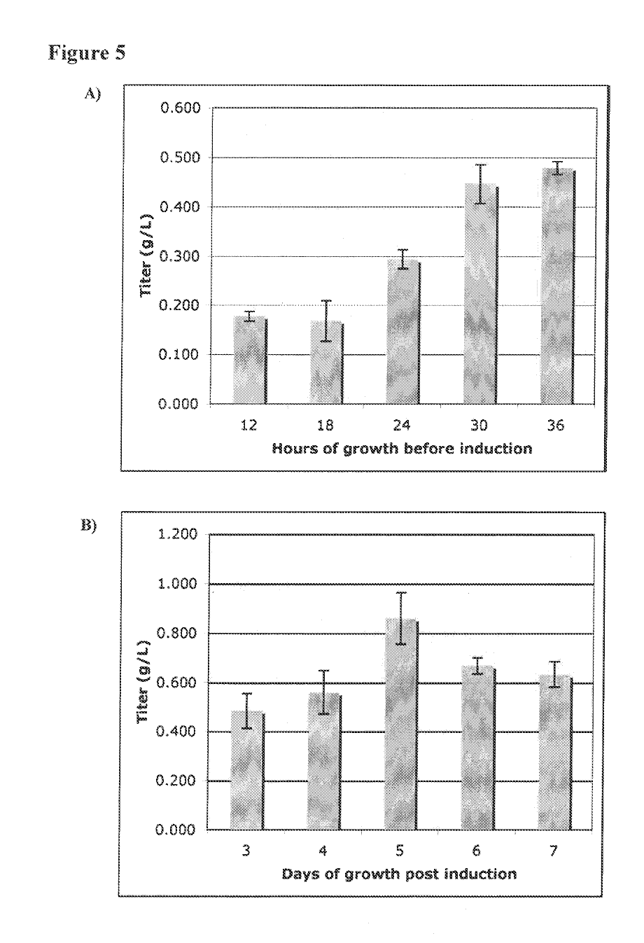

We altered culture conditions in several ways to optimize the titer of compound 5. First, culture time prior to the induction of alcA was investigated. Cultures were incubated from 12 to 36 hours before the chemical inducer cyclopentanone, necessary to induce the alcA promoter, was introduced and thereafter the culture remained under inducing conditions for an additional 72 hours. The experiment revealed the production of 5 was enhanced by growth 30-36 hours before induction (FIG. 5A). We next examined a second parameter, the culture time post induction. The A. nidulans strain was cultured for seven days after induction and samples were collected at one-day intervals from day three through day seven (FIGS. 5A and 5B). An increase and then decline was observed over the period, with the accumulation of 5 peaking on day five. Under optimized expression conditions our engineered strain produce the polyketide (5) abundantly, providing a titer of 900 mg/L. The elevated production of this advanced metabolite allowed us to employ it for the preparation of a small library of azaphilones.

We focused on applying our semisynthetic route to prepare (+)-sclerotiorin by treating 5 with p-toluenesulfonic acid (Scheme 2, FIG. 3) to form the 2-benzopyrilium salt (6), which is then oxidized by lead tetraacetate to generate the non-halogenated azaphilone (7).⁹ Although the acetoxylation at C-7 would be non-stereospecific, the diastereomers were indistinguishable (t.l.c., ¹H and ¹³C NMR) nor can they be separated by HPLC.

Electrophilic chlorination of azaphilone 7 introduces a chlorine atom at C-5 by using a slight excess of N-chlorosuccinimide to provide the natural product (+)-sclerotiorin and 7-epi-sclerotiorin (8) in 61% yield. The diastereomers of 8 were separated by analytical chiral HPLC to reveal close to a 1:1 ratio of (+)-sclerotiorin and 7-epi-sclerotiorin.

Additionally, several azaphilone analogs were also prepared from 5 (Scheme 3, FIG. 4). To create a more efficient route, we were interested in hypervalent-iodine-mediated phenol oxidative dearomatization with o-iodoxybenzoic acid (IBX), a method developed by Pettus and coworkers.¹² The reaction proceeds with the formation of 6, which subsequently is treated with IBX and catalyst Bu₄NI at room temperature to form 11. We observed 14 as the major side product of the reaction. It is plausible that the generation of 14 could arise from tetrabutylammonium triiodide or 10H formed in the presence of the residual acetic acid with adventitious water.^{13,14} To assist in the regioselective halogenation of 11, the corresponding N-halosuccinimides were employed to produce compounds 12, 13 and 14. Then to further functionalize the scaffold of the azaphilone core, a wittig olefination was performed with carbethoxymethylenetriphenylphosphorane. It was observed the glide selectively coupled with the less hindered ketone and produced a mixture of E/Z isomers (1:1.05) as determined by ¹H NMR data. Due to the difficulties in separation, the diasteromeric mixtures of 15-17 were tested towards the inhibition lipoxygenase-1.

Based on a report that (+)-sclerotiorin has potent LOX-1 inhibition,⁵ the biological activities of all semisynthetic azaphilones were evaluated for soybean LOX-1 inhibition and provide a preliminary structure-activity relationship (SAR). In screening for inhibition, azaphilones 7 and 13 displayed the highest lipoxgyenase-1 inhibition (Table 2). Azaphilones 9, 11, and 12 showed similar inhibition activities. Iodinated azaphilones displayed less inhibition toward LOX-1, which may be due, in part to putative chemical instability. The other compounds (15-17), showed no appreciable LOX-1 inhibition.

TABLE 2
Lipoxygenase-1 Inhibitory Activity.

| Compound | IC₅₀ (μM) ± s.d. |
|---|---|
| 1 | 7.8 ± 2.4 |
| 4 | >100 |
| 5 | 97.2 ± 2.0 |
| 7 | 4.9 ± 3.3 |
| 8 | 2.3 ± 0.9 |
| 9 | 6.8 ± 4.5 |
| 10 | 17.4 ± 8.1 |
| 11 | 10.7 ± 6.6 |
| 12 | 7.9 ± 3.9 |
| 13 | 3.2 ± 1.5 |
| 14 | 19.6 ± 11.9 |
| 15 | >100 |
| 16 | >100 |
| 17 | >100 |

The LOX-1 inhibition screening suggested that the C-8 ketone might be an important structural feature for activity against lipoxygenase targets. The azaphilone analogs also indicated that halogenation at C-5 is not essential to maintain low micromolar activity, except that when C-5 was iodinated a loss of inhibition was observed. Since (+)-sclerotiorin has similar IC₅₀ with 8 containing both (+)-sclerotiorin and 7-epi-sclerotiorin, the chiral center C-7 is not critical for the LOX-1 inhibition.

It has been suggested that 1 inhibits LOX-1 by trapping lipid radicals formed at the active site of the enzyme-substrate complex. Although, we have not measured the reductive properties of our semisynthetic azaphilones, it is reasonable that they have similar antioxidant properties to (+)-sclerotiorin.

The metabolic engineering of a biosynthetic pathway in the filamentous fungus, Aspergillus nidulans, demonstrates the feasibility of producing copious amounts of the advanced polyketide (5). Coupled with existing synthetic methodology, this provides facile synthetic access to derivatives of the natural product sclerotiorin. Azaphilone analogs 7 and 13 were the most effective to inhibit the therapeutic target, LOX-1. Preliminary SAR indicates the importance of the C-8 ketone for inhibition of lipoxygenase. This may also provide insight into the further development of more potent LOX-1 inhibitors.

### REFERENCES

1. Brash, A. R. J. Biol. Chem. 1999, 274, 23679.
2. Schneider, C.; Pratt, D. A.; Porter, N. A.; Brash, A. R. Chem. Biol. 2007, 14, 473.
3. Gordon, M. Antioxidants in food, Practical applications; Pokorny, J., Yanishlieva, N., Gordon, M., Eds.; Woodhead Publishing Ltd.: Cambridge, England, 2001; Chapter 2, pp 7.
4. (a) Komoda, T.; Sugiyama, Y.; Abe, N.; Imachi, M.; Hirota, H.; Hirota, A. Tet. Lett. 2003, 44, 1659.
(b) Rao, K. C. S.; Divakar, S.; Rao, A. G. A.; Karanth, N. G.; Suneetha, W. J.; Krishnakantha, T. P.; Sattur, A. P. Biotech. Lett. 2002, 24, 1967.
5. Chidananda, C.; Sattur, A. P. J. Agric. Food Chem. 2007, 55, 2879.
6. Curtin, T. P.; Reilly, J. Biochem J. 1940, 34, 1419.
7. Osmanova, N.; Schultze, W.; Ayoub, N. Phytochem. Rev. 2010, 9, 315.
8. Wei, W.-G.; Yao, Z.-J. J. Org. Chem. 2005, 70, 4585.
9. Chong, R.; King, R. R.; Whalley, W. B. J. Chem. Soc. (C) 1971, 3566.
10. (a) Zhu, J.; Grigoriadis, N. P.; Lee, J. P.; Porco, J. A. J. Am. Chem. Soc. 2005, 127, 9342. (b) Zhu, J.; Porco, J. A. Org. Lett. 2006, 8, 5169. (c) Germain, A. R.; Bruggemeyer, D. M.; Zhu, J.; Genet, C.; O'Brien, P.; Porco, J. A. J. Org. Chem. 2011, 76, 2577.
11. Chiang, Y.-M.; Szewczyk, E.; Oakley, B. R.; Wang, C. C. C. J. Am. Chem. Soc. 2009, 131, 2965.
12. Marsini, M. A.; Gowin, K. M.; Pettus, T. R. R. Org. Lett. 2006, 8, 3481.
13. Perez-Benito, J. F.; Brillas, E.; Arias, C. Can. J. Chem. 1989, 68, 79.
14. Moorthy, J. N.; Senapati, K.; Kumar, S. J. Org. Chem. 2009, 74, 6287.
15. Chiang, Y.-M.; Szewczyk, E.; Oakley, B. R.; Wang, C. C. C. J. Am. Chem. Soc. 2009, 131, 2965.
16. Szewczyk, E.; Nayak, T.; Oakley, C. E.; Edgerton, H.; Xiong, Y.; Taheri-Talesh, N.; Osmani, S. A.; Oakley, B. R. Nat. Protoc. 2006, 1, 3111-3120.
17. Frigerio, M.; Santagostino, M.; Sputore, S. J. Org. Chem. 1999, 64, 4537.
18. Axelrod, B.; Cheesbrough, T. M.; Laakso, S. Meth. Enzymol. 1981, 71, 441.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A method for producing lipoxygenase (LOX) inhibitors comprising:
(i) obtaining a compound 5 having the structure 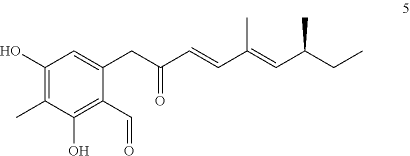 [SMILES: CC[C@H](C)/C=C(C)/C=C/C(=O)Cc1cc(O)c(C)c(O)c1C=O]
(ii) contacting the compound from (i) with p-toluenesulfonic acid to obtain a compound having the structure 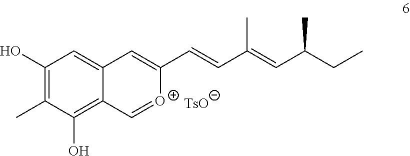 [SMILES: CC[C@H](C)/C=C(C)/C=C/c1cc2cc(O)c(C)c(O)c2c[o+]1]
(iii) contacting the compound from (ii) with an oxidizing agent to obtain a compound having the structure 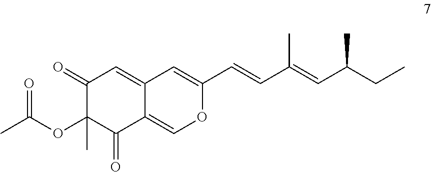 [SMILES: CC[C@H](C)/C=C(C)/C=C/C1=CC2=CC(=O)C(C)(OC(C)=O)C(=O)C2=CO1]
(iv) contacting the compound from (iii) with an N-halosuccinimide to obtain a compound having the structure 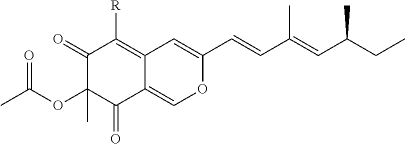
wherein R is Cl, Br or I and wherein the compound produced in step (iv) is LOX inhibitor, so as to produce a LOX inhibitor.

2. A method for producing lipoxygenase inhibitors comprising the steps set forth in FIG. 4 (Scheme 3).

3. The method of claim 1 or 2, wherein compound 5 is obtained from a microbial strain.

4. The method of claim 3, wherein the microbial strain is a mutant strain of Aspergillus nidulans.

5. The method of claim 1 or 2, wherein N-halosuccinimides is any one or more of N-Chlorosuccinimide (NCS), N-Bromosuccinimide (NBS) and N-Iodosuccinimide (NIS).

6. The method of claim 1, wherein the oxidizing agent is lead tetracetate.

7. The method of claim 1 or 2, wherein the lipoxygenase is any one or more of 5-LOX, 8-LOX, 12-LOX and 15-LOX.

8. The method of claim 1 or 2, wherein lipoxygenase inhibitor is an azaphilone.

9. The method of claim 8, wherein an azaphilone is sclerotiorin or analogs thereof.

10. A lipoxygenase inhibitor produced by the method of claim 1 or 2.

11. The lipooxygenase inhibitor of claim 10, wherein the inhibitor is an azaphilone or an analog thereof.

12. The lipoxygenase inhibitor of claim 11, wherein the azaphilone or analog thereof is any one or more of compounds 7, 8, 9, 10, 11, 12, 13 or 14.

13. A pharmaceutical composition comprising the lipoxygenase inhibitor of claim 10, and a pharmaceutically acceptable carrier.

14. A method for treating inflammatory diseases in a subject in need thereof comprising:
(i) providing a composition comprising the lipoxygenase inhibitor of claim 10, and
(ii) administering an effective amount of the composition to the subject, thereby treating inflammatory diseases in the subject.

15. The method of claim 14, wherein the inflammatory disease is any one or more of allergies, asthma, atherosclerosis, cancer, osteoporosis.
